(19) United States
(12) Patent Application Publication
(10) Pub. No.: US 2005/0256178 A1
(43) Pub. Date: Nov. 17, 2005

(54) Novel nutraceutical compositions comprising boitin

(76) Inventors: Manfred Ludwig Eggersdorfer, Stein (CH); Daniel Raederstorff, Brunstatt (FR); Sandra Renata Teixeira, Arlington, MA (US); Peter Weber, Malsburg-Marzell (DE)

Correspondence Address: Stephen M Haracz;Bryan Cave, 1290 Avenue of the Americas, New York, NY, 10104, US

(21) Appl. No.: 10/525,348

(22) PCT Filed: Aug. 18, 2003
(86) PCT No.: PCT/EP03/09112

§ 371 Date: Feb. 22, 2005

(30) Foreign Application Priority Data
Aug. 23, 2002 (EP) 02018847.0
Jun. 26, 2003 (EP) 03014625.2

(51) Int. Cl. A61K 31/4188; A61K 31/385; A61K 31/198; A61K 31/045

(52) U.S. Cl. 514/393; 514/440; 514/561; 514/724

(57) ABSTRACT

Nutraceutical compositions comprise biotin in an amount sufficient to administer to a subject a daily dosage of 0.01 mg per kg body weight to about 3 mg per kg body weight and at least one additional component selected from pantethine or a metabolite thereof, EGCG, phytanic acid, lipoic acid and policosanol. The compositions are useful for the treatment of both type 1 and 2 diabetes, and for the prevention of type 2 diabetes in those individuals with pre-diabetes, or impaired glucose tolerance (IGT) or obesity.

The present invention relates to novel nutraceutical compositions comprising biotin as the active ingredient for the treatment or prevention of diabetes mellitus, or other conditions associated with impaired glucose tolerance such as syndrome X and obesity, and at least one additional component selected from pantethine or a metabolite thereof, EGCG, phytanic acid, lipoic acid and policosanol. In one aspect the present invention relates to compositions comprising biotin in an amount sufficient to administer to a subject a daily dosage of 0.01 mg per kg body weight to about 3 mg per kg body weight, and at least one additional component selected from pantethine or a metabolite thereof, EGCG, phytanic acid, lipoic acid and policosanol, and to the use of such compositions as a nutritional supplement for the said treatment or prevention, e.g., as an additive to a multi-vitamin preparations comprising vitamins and minerals which are essential for the maintenance of normal metabolic function but are not synthesized in the body. In a further aspect, the present invention relates to such biotin compositions and their use wherein the additional component(s) is (are) selected from pantethine or a metabolite thereof, EGCG, phytanic acid and lipoic acid.

The compositions of the present invention are particularly intended for the treatment of both type 1 and 2 diabetes, and for the prevention of type 2 diabetes in those individuals with pre-diabetes, or impaired glucose tolerance (IGT), or obesity.

The compositions comprising a combination of active ingredients, i.e., biotin and at least one additional component selected from pantethine or a metabolite thereof, EGCG, phytanic acid, lipoic acid and policosanol have different mechanism of action on glucose metabolism and insulin sensitivity thus providing additive and/or synergetic effects in the treatment of diabetes.

The term nutraceutical as used herein denotes a usefulness in both the nutritional and pharmaceutical field of application. Thus, the novel nutraceutical compositions can find use as supplement to food and beverages, and as pharmaceutical formulations for enteral or parenteral application which may be solid formulations such as capsules or tablets, or liquid formulations, such as solutions or suspensions. As will be evident from the foregoing, the term nutraceutical composition also comprises food and beverages containing biotin and at least one additional component selected from pantethine or a metabolite thereof, EGCG, phytanic acid, lipoic acid and policosanol, as well as supplement compositions containing the aforesaid active ingredients.

Diabetes is a widespread chronic disease that hitherto has no cure. The incidence and prevalence of diabetes is increasing exponentially and it is among the most common metabolic disorder in developed and developing countries. Diabetes mellitus is a complex disease derived from multiple causative factors and characterized by impaired carbohydrate, protein and fat metabolism associated with a deficiency in insulin secretion and or insulin resistance. This results in elevated fasting and postprandial serum glucose that leads to complications if left untreated. There are two major categories of the diseases, insulin-dependent diabetes mellitus (IDDM, type 1) and non-insulin-dependent diabetes mellitus (NIDDM, type 2).

Type 1 and type 2 diabetes are associated with hyperglycemia, hypercholesterolemia and hyperlipidemia. The insensitivity to insulin and absolute insulin deficiency in type 1 and2 diabetes leads to a decrease in glucose utilization by the liver, muscle and the adipose tissue and to an increase in the blood glucose levels. Uncontrolled hyperglycemia is associated with increased and premature mortality due to an increased risk for microvascular and macrovascular diseases, including nephropathy, neuropathy, retinopathy, hypertension, stroke, and heart disease. Recent evidence showed that tight glycemic control is a major factor in the prevention of these complications in both type 1 and type 2 diabetes mellitus. Therefore, optimal glycemic control by drugs or therapeutic regimens is an important approach for the treatment of diabetes.

Therapy of type 2 diabetes initially involves dietary and lifestyle changes, when these measures fail to maintain adequate glycemic control the patients are treated with oral hypoglycemic agents and/or exogenous insulin. The current oral pharmacological agents for the treatment of type 2 diabetes mellitus include those that potentiate insulin secretion (sulphonylurea agents), those that improve the action of insulin in the liver (biguanide agents), insulin sensitizing agents (thiazolidinediones) and agents which act to inhibit the uptake of glucose (α-glucosidase inhibitors). However, currently available agents generally fail to maintain adequate glycemic control in the long term due to progressive deterioration in hyperglycaemia, resulting from progressive loss of pancreatic cell function. The proportion of patients able to maintain target glycemic levels decreases markedly overtime necessitating the administration of additional/alternative pharmacological agents. Furthermore, the drugs may have unwanted side effects and are associated with high primary and secondary failure rates. Finally, the use of hypoglycemic drugs may be effective in controlling blood glucose levels, but may not prevent all the complications of diabetes. Thus, current methods of treatment for all types of diabetes mellitus fail to achieve the ideals of normoglycemia and the prevention of diabetic complications.

Therefore, although the therapies of choice in the treatment of type 1 and type 2 diabetes are based essentially on the administration of insulin and of oral hypoglycemic drugs, there is a need for a safe and effective nutritional supplement with minimal side effects for the treatment and prevention of diabetes. Many patients are interested in alternative therapies which could minimize the side effects associated with high-dose of drugs and yield additive clinical benefits. Patients with diabetes have a special interest in treatment considered as “natural” with mild anti-diabetic effects and without major side effects, which can be used as adjuvant treatment. Type 2 diabetes is a progressive and chronic disease, which usually is not recognized until significant damage has occurred to the pancreatic cells responsible for producing insulin. Therefore, there is also an increasing interest in the development of a dietary supplement that may be used to prevent the development of diabetes in people at risk especially in elderly who are at high risk for developing diabetes. Furthermore, type 2 is a complicated disease resulting from coexisting defects at multiple organ sites: resistance to insulin action in muscle and adipose tissues, defective pancreatic insulin secretion, unrestrained hepatic glucose production associated with lipid abnormalities and endothelial dysfunction. Therefore, given the multiple pathophysiological lesions in type 2 diabetes, combination therapy is an attractive approach to its management.

The use of biotin in daily dosages of about 0.01 mg per kg body weight to about 3 mg per kg body weight, particularly in specific combinations with pantethine or a metabolite thereof, EGCG and/or phytanic acid which individually exert different mechanisms of action are effective in achieving and maintaining target blood glucose levels in diabetic patients.

The combinations of the active ingredients identified above are preferred because of their different actions, to take advantage of additive/synergetic and multiorgan effects. Owing to distinct mechanism of action of the individual active ingredients the combinations not only improve glycemic control, but also result in lower drug dosing in some settings and minimize adverse effects. Because of their distinct mechanism and sites of action, the specific combinations of dietary supplements discussed above also take advantage of additive/synergetic effects to achieve a degree of glucose lowering greater than single agents can accomplish. Thus, although the therapies of choice in the therapeutic treatment of type 1 and type 2 diabetes is based essentially on the administration of insulin and of oral hypoglycemic drugs appropriate nutritional therapy is also of major importance for the successful treatment of diabetics.

The function of each of the active ingredients of the nutraceutical compositions of the present invention is described below:

Biotin: Biotin supplementation enhances hepatic glucose clearance which results in a decrease of circulating glucose concentration and induces decrease in the hepatic PEPCK activity. PEPCK is a rate-limiting cytosolic enzyme that catalyses the first committed step of hepatic gluconeogenesis. Decrease of hepatic PEPCK activity results in a decrease in liver glucose output. In accordance with the invention it has been found that biotin given orally (2-16 mg/day) or parentally (0.1 mg/day) improved oral glucose tolerance in diabetic KK mice (NIDDM model), streptozotocin-diabetic rats (IDDM) and pre-diabetic Otsuka Long-Evans Tokushima fatty (OLETF) rats (NIDDM). Preliminary human studies showed that after biotin supplementation fasting blood glucose levels decreased in type 1 and type 2 diabetic patients.

Thus, high doses of biotin may improve hyperglycemia in type 1 and type 2 diabetic patients. Biotin decreases hepatic glucose output and benefits glucose-stimulated insulin secretion. A combination of biotin with a product improving peripheral insulin sensitivity is, therefore, valuable in diabetes management. Such products are, particularly, phytanic acid and lipoic acid.

EGCG: Epigallocatechin gallate (EGCG) is the major catechin found in green tea. In rats green tea catechins dose-dependently suppressed the increase in glucose and insulin levels in plasma after a starch or a sucrose rich meal. Combinations of biotin and EGCG according to the invention are especially useful for patients who have impaired glucose tolerance, older patients who develop an increase in postprandial glucose due to aging, and patients with undiagnosed diabetes.

Pantethine: In human studies oral administration of pantethine resulted in a progressive decrease in total cholesterol, triglycerides, low density lipoprotein (LDL) cholesterol and an increase in high density lipoprotein (HDL) cholesterol. Thus, resulting in a more favorable Chol/HDL ratio which reduces cardiovascular risk. Diabetes mellitus is associated with a 3- to 4-fold increase in risk of coronary artery disease. Type 2 diabetes mellitus adversely affects the plasma lipid profile, increasing levels of atherogenic lipids such as low density lipoproteins (LDL) and very low density lipoproteins (VLDL), but decreasing levels of high density lipoprotein (HDL), an antiatherogenic lipid. Atherosclerotic manifestations are not only common in individuals with diabetes but also result in significant long-term complications. Therefore, the oral supplementation with pantethine helps diabetes patients to normalize their lipid values reducing the risk of coronary heart disease and of thrombotic events. Instead of or in addition to panthethine, metabolites of pantethine such as cysteamine may find use in accordance with the invention.

Lipoic acid: Lipoic acid (1,2-dithiolane-3-pentaenoic acid) plays an essential role in mitochondrial-specific pathways that generate energy from glucose and may potentially influence the rate of glucose oxidation. Lipoic acid stimulates glucose transport in both muscle and adipose cells in culture. Moreover, administration of lipoic acid also raised basal and insulin-stimulated glucose uptake by skeletal muscles of glucose intolerant and non-insulin dependent diabetic animals. Furthermore, lipoic acid improves glucose disposal in patients with type 2 and may be incorporated in a nutraceutical composition of the present invention in order to prevent and/or treat the diabetic related complications and as agent with insulin sensitizing activity.

Phytanic acid: Phytanic acid (3,7,11,15-tetramethylhexadecanoic acid) at concentrations ranging from about 10 to about100 μM enhances uptake of glucose in rat primary hepatocytes. Compared to the specific PPAR-γ agonist such as ciglitazone, phytanic acid exerts only minor effects on the differentiation of pre-adipocyte cells into mature adipocytes. Therefore, intake of phytanic acid helps to improve insulin sensitivity and may act as a preventative measure against type 2 diabetes and Syndrome X through activation of PPARs and RXR.

Policosanol: Policosanol is a mixture of primary aliphatic alcohols isolated and purified from plant waxes, mainly sugar cane. The aliphatic alcohol of the mixture is a CH₃-(CH₂)ₙ—CH₂OH alcohol with chain length varying from 18 to 40 carbon atoms. Typical aliphatic alcohols of the mixture are octacosanol, hexacosanol, heptacosanol, triacontanol and dotriacontanol. Policosanol has been shown to lower cholesterol in animal models, healthy volunteers, and patients with type II hypercholesterolemia. Therefore, it is useful in the dyslipidemia associated with type 2 diabetes mellitus.

A multi-vitamin and mineral supplement may be added to the nutraceutical compositions of the present invention to obtain an adequate amount of an essential nutrient missing in some diets. The multi-vitamin and mineral supplement may also be useful for disease prevention and protection against nutritional losses and deficiencies due to lifestyle patterns and common inadequate dietary patterns sometimes observed in diabetes. Moreover, oxidant stress has been implicated in the development of insulin resistance. Reactive oxygen species may impair insulin stimulated glucose uptake by disturbing the insulin receptor signaling cascade. The control of oxidant stress with antioxidants such as α-tocopherol (vitamin E) ascorbic acid (vitamin C) may be of value in the treatment of diabetes. Therefore, the intake of multi-vitamin supplement may be added to the above mentioned active substances to maintain a good balanced nutrition.

The nutraceutical composition of the present invention contains biotin in an amount sufficient to administer to a subject a dosage from about 0.01 mg to about 3 mg per kg body weight per day, preferably from about 0.1 mg to about 0.5 mg per kg body weight per day. Thus, if the nutraceutical composition is a food or beverage the amount of biotin contained therein is suitably in the range from about 0.03 mg per serving to about 50 mg per serving. If the nutraceutical composition is a pharmaceutical formulation such formulation may contain from about 0.35 mg to about 200 mg per solid dosage unit, e.g., per capsule or tablet, or a corresponding dosage in a liquid formulation, or from about 0.35 mg per daily dose to about 200 mg per daily dose.

In a preferred aspect of the invention, the nutraceutical composition of the present invention further contains pantethine. The amount of pantethine in the composition may be such to provide a daily dosage from about 1 mg per kg body weight to about 50 mg per kg body weight of the subject to which it is to be administered. A food or beverage suitably contains about 20 mg per serving to about 800 mg per serving of pantethine. If the nutraceutical composition is a pharmaceutical formulation such formulation may contain pantethine in an amount from about 20 mg to about 1000 mg per dosage unit, e.g., per capsule or tablet, or from about 70 mg per daily dose to about 3500 mg per daily dose of a liquid formulation.

If EGCG is present in the composition according to the invention its amount may be such to provide a daily dosage from about 0.3 mg per kg body weight to about 30 mg per kg body weight of the subject to which it is to be administered. A food or beverage suitably contains about 5 mg per serving to about 500 mg per serving of EGCG. If the nutraceutical composition is a pharmaceutical formulation such formulation may contain EGCG in an amount from about 10 mg to about 500 mg per dosage unit, e.g., per capsule or tablet, or from about 20 mg per daily dose to about 2000 mg per daily dose of a liquid formulation.

If phytanic acid is present in the nutraceutical composition according to the invention its amount may be such to provide a daily dosage from about 1 mg per kg body weight to about 100 mg per kg body weight of the subject to which it is to be administered. A food or beverage suitably contains about 20 mg per serving to about 2000 mg per serving of phytanic acid. If the nutraceutical composition is a pharmaceutical formulation such formulation may contain phytanic acid in an amount from about 30 mg to about 500 mg per dosage unit, e.g., per capsule or tablet, or from about 70 mg per daily dose to about 7000 mg per daily dose of a liquid formulation. Phytanic acid may also be used in the form of a biologically equivalent derivative thereof, such as an ester, e.g. the methyl or ethyl ester.

If lipoic acid is present in the nutraceutical composition according to the invention its amount may be such to provide a daily dosage from about 0.3 mg per kg body weight to about 30 mg per kg body weight of the subject to which it is to be administered. A food or beverage suitably contains about 5 mg per serving to about 500 mg per serving of lipoic acid. If the nutraceutical composition is a pharmaceutical formulation such formulation may contain lipoic acid in an amount from about 5 mg to about 800 mg per dosage unit, e.g., per capsule or tablet, or from about 5 mg per daily dose to about 2000 mg per daily dose of a liquid formulation.

If policosanol is present in the nutraceutical composition according to the invention its amount may be such to provide a daily dosage from about 0.002 mg per kg body weight to about 1.5 mg per kg body weight of the subject to which it is to be administered. A food or beverage suitably contains about 0.1 mg per serving to about 20 mg per serving of policosanol. If the nutraceutical composition is a pharmaceutical formulation such formulation may contain policosanol in an amount from about 0.1 mg to about 30 mg per dosage unit, e.g., per capsule or tablet, or from about 0.1 mg per daily dose to about 100 mg per daily dose of a liquid formulation.

The nutraceutical compositions of the present invention preferably comprise combinations of

Biotin and pantethine. Also preferred are compositions comprising

Biotin and phytanic acid;
Biotin and EGCG;
Biotin and lipoic acid;
Biotin, phytanic acid and EGCG;
Biotin, phytanic acid and pantethine;
Biotin, pantethine and EGCG; and
Biotin, phytanic acid, pantethine and EGCG.
   Dosage Ranges (for a 70 kg person)
Biotin: 0.7 to 210 mg /day
EGCG: 20-2100 mg/day
Pantethine: 70-3500 mg/day
Phytanic acid: 70-7000 mg/day
Lipoic acid: 20-2100 mg/day
Policosanol: 0.15-100 mg/day

The following Examples illustrate the invention further.

A. Pharmaceutical compositions may be prepared by conventional formulation procedures using the ingredients specified below:

### EXAMPLE 1

### Soft Gelatin Capsule

Soft gelatin capsules are prepared by conventional procedures using ingredients specified below:
Active ingredients: Biotin 30 mg Pantethine 100 mg
Other ingredients: glycerol, water, gelatine, vegetable oil

### EXAMPLE 2

### Hard Gelatin Capsule

Hard gelatin capsules are prepared by conventional procedures using ingredients specified below:
Active ingredients: Biotin 30 mg Pantethine 100 mg
Other ingredients:
Fillers: lactose or cellulose or cellulose derivatives q.s
Lubricant: magnesium sterate if necessary (0.5%)

### EXAMPLE 3

### Tablet

Tablets are prepared by conventional procedures using ingredients specified below:
Active ingredients: Biotin 20 mg, pantethine 50 mg
Other ingredients: microcrystalline cellulose, silicone dioxide (siO2), magnesium stearate, crosscarmellose sodium.

B. Food items may be prepared by conventional procedures using ingredients specified below:

### EXAMPLE 4

### Soft Drink with 30% Juice

Active ingredients:
Biotin and, optionally, one or more aditional components selected from pantethine, EGCG, phytanic acid, lipoic acid and policosanol are incorporated in this food item
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving
Typical serving: 240 ml

I. A Soft Drink Compound is prepared from the following ingredients:

Juice Concentrates and Water Soluble Flavours

| | [g] |
|---|---|
| Orange concentrate | |
| 60.3° Brix, 5.15% acidity | 657.99 |
| Lemon concentrate | |
| 43.5° Brix, 32.7% acidity | 95.96 |
| Orange flavour, water soluble | 13.43 |
| Apricot flavour, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavours | |
| Orange flavour, oil soluble | 0.34 |
| Orange oil distilled | 0.34 |

1.6 Active Ingredients

Active ingredients (this means the active ingredient mentioned above: biotin and one or more of the following EGCG, pantethine, lipoic acid and/or phytanic acid) in the concentrations mentioned above.

Fruit juice concentrates and water soluble flavours are mixed without incorporation of air. The color is dissolved in deionized water. Ascorbic acid and citric acid is dissolved in water. Sodium benozoate is dissolved in water. The pectin is added unter stirring and dissolved while boiling. The solution is cooled down. Orange oil and oil soluble flavours are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenising using a Turrax and then a high-pressure homogenizer (p₁=200 bar, p₂=50 bar).

II. A Bottling Syrup is prepared from the following ingredients:

| | [g] |
|---|---|
| Softdrink compound | 74.50 |
| Water | 50.00 |
| Sugar syrup 60° Brix | 150.00 |

The ingredients of the bottling syrup are mixed together. The bottling syrup is diluted with water to 1 l of ready to drink beverage.

Variations:

Instead of using sodium benzoate, the beverage may be pasteurised. The beverage may also be carbonised.

### EXAMPLE 5

### 5 Cereal Bread

Active ingredients:
Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 50 g

| | [%] |
|---|---|
| 5 cereal flour | 56.8 |
| Water | 39.8 |
| Yeast | 2.3 |
| Salt | 1.1 |

The yeast is dissolved in a part of the water. All ingredients are mixed together to form a dough. Salt is added at the end of the kneading time. After fermentation, the dough is reworked and divided before a loaf is formed. Before baling, the surface of the loaf is brushed with water and sprinkled with flour.

Procedure Parameters:

Kneading:

| | |
|---|---|
| Spiral kneading system | 4 min 1^{st} gear; 5 min 2^{nd} gear |
| Dough proofing: | 60 min |
| Dough temperature: | 22-24° C. |
| Proofing time: | 30 min |

Baking:

| | |
|---|---|
| Oven: | Dutch type oven |
| Baking temperature: | 250/220° C. |
| Baking time: | 50-60 min |

### EXAMPLE 6

### Cookies Type Milano

Active ingredients:
Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 30 g

| | [g] |
|---|---|
| Wheat Flour, type 550 | 41.0 |
| Sugar | 20.5 |
| Fat/Butter | 20.5 |
| Whole egg (liquid) | 18.0 |
| Lemon Flavour | q.s. |
| Baking agent | q.s. |

All ingredients are added slowly under mixing to form a sweet short pastry.

Afterwards, the pastry is kept cool (4° C.) for at least 2 hours before flattening the pastry to a thickness of approx. 5 mm. Pieces are cut out and brushed with egg yolk on the surface before baking.

Baking:

| | |
|---|---|
| Oven: | fan oven |
| Baking temperature: | 180° C. |
| Baking time: | 15 min |

### EXAMPLE 7

### Toast

Active ingredients:
Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 100 g

| | [%] |
|---|---|
| Wheat Flour, type 550 | 55.4 |
| Water | 33.2 |
| Yeast | 2.8 |
| Salt | 1.1 |
| Fat/Butter | 5.5 |
| Malt | 0.6 |
| Emulsifier baking agent | 1.4 |

The yeast is dissolved in a part of the water. All ingredients are mixed together to form a dough. Salt is added at the end of the kneading time. Afterwards, the dough is reworked, divided and placed in a baking tin for fermentation. After baking, the loaf is unmoulded directly.

Process Parameters:

Kneading:

| | |
|---|---|
| Spiral kneading system | 5-6 min 1^{st} gear; 3-4 min 2^{nd} gear |
| Dough proofing: | none |
| Dough temperature: | 22-24° C. |
| Proofing time: | 40 min |

Baking:

| | |
|---|---|
| Oven: | Dutch type oven |
| Baking temperature: | 220° C. |
| Baking time: | 35-40 min |

### EXAMPLE 8

### Yoghurt—Set Type, 3.5% Fat

Active ingredients:
Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 225 g

| | [%] |
|---|---|
| Full fat milk (3.8% fat) | 90.5 |
| Skimmed milk powder | 2.0 |
| Sugar | 5.0 |
| Culture | 2.5 |

The milk is heated to 35° C. before addition of milk powder, stabiliser, sugar and active ingredients. This mixture is heated to 65° C. to dissolve all ingredients. Then the mixture is homogenized in a high-pressure homogenizer (p₁=150 bar, p₂=50 bar) at 65° C. This emulsion is then pasteurised at 80° C. for 20 minutes. After cooling to 45° C. natural yoghurt/culture is added and mixed. Then this mixture is filled into cups and fermented at 45° C. for 3-4 hours until a pH of 4.3 is reached and then stored at 4° C.

### EXAMPLE 9

### Yoghurt—Stirred Type, 3.5% Fat

Biotin and, optionally, one or more additional components selected from pantethine, EGCG, phytanic acid, lipoic acid and policosanol are incorporated in this food items:
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Tropical serving: 225 g

| | [%] |
|---|---|
| Full fat milk (3.8% fat) | 90.2 |
| Skimmed milk powder | 2.0 |
| Stabiliser | 0.3 |
| Sugar | 5.0 |
| Culture | 2.5 |

The milk is heated to 35° C. before addition of milk powder, stabiliser, sugar and active ingredients. This mixture is heated to 65° C. to dissolve all ingredients before homogenisation in a high-pressure homogenizer (p₁=150 bar, p₂=50 bar) at 65° C. This emulsion is then pasteurised at 80° C. for 20 minutes. After cooling to 45° C. natural yoghurt/culture is added and mixed, followed by fermentation at 45° C. for 3-4 hours until a pH of 4.3 is reached. After cooling and stirring vigorously, the yoghurt is filled in cups and stored at 4° C.

### EXAMPLE 10

### Ice cream, 8% Fat

Active ingredients:

Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 85 g

| | [g] |
|---|---|
| Milk (3.7% fat) | 600.00 |
| Cream (35% fat) | 166.00 |
| Skim milk powder | 49.10 |
| Sugar | 109.00 |
| Glucose syrup 80% | 70.00 |
| Ice cream stabiliser | 5.00 |
| Flavor | q.s. |

Colorq.s

Sugar, skim milk powder and stabiliser are added to the milk and cream, mixed and heated to 45° C. Then the colour as stock solution and the glucose syrup is added as well as the active ingredients. The mix is heated up and pasteurized (20 min, 80° C.). Then a homogenization step takes place. Afterwards the mix is cooled down under constant stirring and the flavour is added at 5° C. The Mix maturated at 5° C. during at least 4 h and then passed through an the ice cream machine (overrun ca. 100%). The ice cream is filled into cups and stored at −20 to −30° C.

### EXAMPLE 11

### Wine Gums

Active ingredients:
Biotin and one or more additional components selected from pantethine, EGCG, phytanic acid, lipoic acid and policosanol are incorporated in this food items
Biotin: 0.03-50 mg/per serving
Pantethine: 20-800 mg/per serving
EGCG: 5-500 mg/per serving
Phytanic acid: 20-2000 mg/per serving
Lipoic acid: 5-500 mg/per serving
Policosanol: 0.1-20 mg/per serving

Typical serving: 30 g

| | [g] |
|---|---|
| Gelatine 200 Bloom | 80.0 |
| Water I | 125.0 |
| Sugar crys. | 290.0 |
| Water II | 120.0 |
| Glucose-syrup DE 38 | 390.0 |
| Citric acid | 10.0 |
| Flavour | 2.0 |
| Colour | q.s. |
| Yield ca | 1000.0 |

Disperse gelatine in water I, stir and dissolve by heating over a stream bath or using a microwave. Mix sugar with water II and bring to boiling until a clear solution is obtained. Remove from heat source. Mix with glucose syrup while dissolved sugar solution is still hot. Slowly add the gelatine solution. Let rest until foam on surface can be removed and 60-65° C. is reached. Add flavour, citric acid and the colour solution as well as active ingredients under stirring. Deposit into moulds printed into starch trays and let sit for at least 48 hours at room temperature. Remove starch powder and polish with oil or wax. Dry at room temperature and package into airtight pouches.

### EXAMPLE 12

The efficacy of the combination of biotin and phytanic acid as well as of both compounds alone on glucose removal was tested in a 5-week study in C57BLKS/J db/db mice (n=7−8/group). This model of late type 2 diabetes with severe hyperglycemia is widely used to determine the efficacy of anti-diabetic compounds.

Male db/db mice were obtained from Jackson Laboratory (Bar Harbor, Me., USA). Adult mice aged 8 weeks were used in the experiment. Mice were housed individually in plastic cages with bedding and allowed free access to standard rodent food and tap water. The animal rooms were controlled for temperature (24° C.), humidity (55%), and light (12-h light-dark cycle). The animals were randomized into four groups. Biotin and phytanic acid were administered as feed-ad-mix. Corn oil (1% of diet) served as a carrier substance for biotin and phytanic acid as well as a placebo when used alone. Group 1 received placebo, group 2 received biotin at a dose of 8 mg/kg body weight (BW)/day, group 3 received phytanic acid at a dose of 300 mg/kg BW/day, and group 4 received the combination of biotin and phytanic acid at a doses of 8 and 300 mg/kg BW/day, respectively. After 4 weeks of treatment glucose removal was assessed 90, 120, 150 and 180 minutes after an oral glucose load (1 g/kg body weight). Blood samples were obtained 90, 120, 150, 180 minutes from the tail vein for determination of blood glucose levels. Blood glucose was measured by a glucose analyzer (Glucotrend Premium, Roche Diagnostics, Rotkreuz, Switzerland). All data are expressed as means for animals in each diet group. Statistical significance of the mean differences between dietary groups was tested by one-way analysis of variance (ANOVA). If significant differences were found, the Dunnett's test for multiple comparison was used to compare each group to the control group. P values less than 0.05 were considered significant. All analyses were performed with Statistica (ver. 5.5A, StatSoft, Inc).

During the glucose load (1 g glucose/kg body weight) the blood glucose levels of animals treated with the combination of biotin and phytanic acid were lower at all time points when compared to the control group. Neither biotin nor phytanic acid caused a significant decrease in the glucose levels when used as a monotherapy.

The combined treatment with biotin and phytanic acid exerted an unexpected synergistic effect on the glucose removal rate (GRR). GRR is defined as the speed at which glucose is removed from blood and directed to the peripheral tissues. At 90, 120, 150 and 180 minutes after application of the glucose load blood glucose levels in animals treated with the combination of biotin and phytanic acid decreased to a greater extent than excepted by the decrease due to monotherapy with biotin or phytanic acid. These facts indicate that GRR is synergistically enhanced in animals treated with the combination of biotin and phytanic acid. To evaluate the GRR the glucose levels were expressed as the percent change of glucose levels for each group compared to the control group. Values of GRR are given in Table 1. The expected GRR is defined as the sum of the GRR exerted by biotin and by phytanic acid when used as a monotherapy.

TABLE 1
Glucose removal rate (% glucose decrease of control) at 90,
120, 150, and 180 minutes of an OGTT in db/db mice treated
with biotin, phytanic acid, and the combination of both compounds.

| | Glucose removal rate (%) | | | |
|---|---|---|---|---|
| | 90 min | 120 min | 150 min | 180 min |
| Biotin (8 mg/kg BW/day) | 11.6 | 10.2 | 15.3 | 15.0 |
| Phytanic acid | 9.7 | 12.5 | 14.6 | 15.2 |
| (300 mg/kg BW/day) | | | | |
| Expected: (Biotin (8 mg/kg | 21.3 | 22.7 | 29.9 | 30.2 |
| BW/day) + Phytanic acid | | | | |
| (300 mg/kg BW/day)) | | | | |
| Found: Biotin (8 mg/kg BW/day) + | 25.9* | 31.0* | 35.1* | 33.2* |
| Phytanic acid (300 mg/kg BW/day) | | | | |

*significantly different from control (p values less than 0.05)

Table 1 shows that the effect in the group supplemented with the combination of biotin and phytanic acid is greater than the sum of the effects of the groups receiving biotin and phytanic acid alone. Thus, the combination of biotin and phytanic acid has a synergetic effect on glucose metabolism.

### EXAMPLE 13

The effect of botin in combination with the compounds EGCG and cysteamin was investigated on the synergistic regulation of genes involved in liver glucose metabolism. The Affymetrix GeneChip® high-density oligonucleotide microarray approach was chosen to determine the global gene expression in H-4-II-E rat liver cells. Those genes that showed synergistic behaviour in their mode of regulation in one of the combination treatment conditions were filtered out and glucose homeostasis marker genes were selected for further analysis. Liver carbohydrate metabolism is tightly regulated. Two specific enzymes, glucokinase (GK) and glucose-6 phosphatase (Glc6Pase), enable the liver to play a crucial role in glucose homeostasis. Since excessive production of glucose is the major cause of fasting hyperglycemia and diabetes mellitus in humans, modulation of the hydrolysis of glucose-6-phosphate by Glc-6-Pase is the distal rate-determining enzymatic step in the process of releasing glucose into the circulation. A marked increase of hepatic Glc-6-Pase mRNA levels has been reported in diabetic animal models. Therefore compounds or combinations of compounds that could reduce expression of the catalytic subunit of the Glc-6-Pase could be considered to normalize hyperglycemia and prevent the diabetic state. EGCG has previously been shown to decrease Glc-6-Pase mRNa levels in H-4-II-E cells. It is also known that biotin could induce the expression of GK in rat hepatocytes.

Cell Culture

H-4-II-E rat liver cells were obtained from the American Type Culture Collection (ATCC) and cultured in Medium 199 (Invitrogen) Basel, Switzerland) supplemented with 10% fetal calf serum in a humidified 5% CO₂ atmosphere at 37° C. Cells were regularly passaged at subconfluence and used at low passage numbers. For the final assays, cells were trypsinized, seeded at a density of 1×10⁶ cells/well in 6-well cell culture plates and maintained in Medium 199 & 0.1% BSA (Invitrogen) for another 6 h before compounds were applied. EGCG was applied in DMSO; cysteamin was first solved in 1M HCl and then applied to the stimulation medium, biotin was directly dissolved into the medium. After 24 h treatment, total RNA was harvested.

High-Density Oligonucleotide Array Hybridization

A total number of 24 samples were prepared following the Affymetrix GeneChip® array protocol (Affymetrix, Santa Clara, Calif., USA). Briefly, total cellular RNA was extracted by using Qiagen RNeasy Mini Kit with an on-column RNase-free DNase I digest (Qiagen, Basel, Switzerland). A T7-(T)₂₄ primer (5′-GGCCAGTGAATTGTAATACGA CTCACTATAGGGAGGCGG-(dT)₂₄-3′) was annealed to 10 kg of total RNA and Superscript II reverse transcriptase (400 U) was utilized to synthesize first-strand cDNA in the presence of DTT, dNTPs and 1× reaction buffer. Second strand synthesis was performed by adding E. coli DNA polymerase I (40 U), E. coli ligase (10 U) and RNase H (2 U) in a final reaction containing 1× second strand buffer in the presence of dNTPs. Finally strands were blunted using T4 DNA polymerase (10 U) (Superscript™ Microarray Customized Kit, Invitrogen, Basel, CH). cDNA was purified by phenol/chloroform extraction and subsequently in vitro transcription was carried out for 3 h using T7 RNA polymerase (Megascript™ T7 Kit, Ambion, Tex., USA), incorporating bio-16-UTPs and bio-11-CTPs (Roche Molecular Biochemicals, Penzberg, Germany). After RNeasy purification, 10 g of the resulting cRNA was fragmented using 40 mM Tris acetate (pH 8.1), 100 mM potassium acetate and 30 mM magnesium acetate at 95° C. for 35 min. A hybrydization cocktail was prepared containing 100 mM MES buffer, 1M NaCl, 20 mM EDTA, 0.01% Tween 20, the sample cRNA, fragmented bacterial control spikes, the biotinylated oligo 984, herring sperm DNA (0.5 μg/μl; Invitrogen) and acetylated BSA (0.25 μg/μl; Promega, Madison, Wis., USA) as described in the Affymetrix GeneChip® Expression Analysis Technical Manual. Samples were then hybridized onto Affymetrix Genechip® Rat 230 (SubA) for 16 h at 45° C. Finally arrays were washed in the GeneChip® Fluidics 400 station (Affymetrix) with the EukGE-WS2v3 program and staining was carried out twice with streptavidinR-phycoerythrin (SAPE) using an antibody amplification protocol.

Data Analysis

Raw fluorescence data were collected by confocal laser scanning (Hewlett Packard, Palo Alto, Calif., USA) and analyzed with the Affymetrix Microarray Suite (MAS 4.0). Data processing was carried out using the RACE-A analysis tool (Roche, Basel, Switzerland). All arrays were normalized against the mean of the total sums of Average Difference (AvgDiff) values across all used arrays. Normalized AvgDiff values below 4 were automatically assigned to a value of 4. Mean average difference values (MeanAvgDiff) and standard deviations (SD) were calculated from the replicate samples. Treatment groups were vehicle (V; 0.1% DMSO); 50 μM EGCG (A), 1 μM biotin (B), 50 μM cysteamin (C), applied as single compounds, as well as the combinations of EGCG/biotin (D), and cysteamin/biotin (E); each experiment was performed in triplicate. For a comprehensive analysis genes with a maximal synergistic factor (SF)>+/−1, combined with a significance level in a unpaired t-test of p<0.05 were filtered. Relative gene expression within a treatment group is equal to the mean average difference (MeanAvgDiff). As the experiment is done in replicate, the corresponding mean values are \overline{X}, \overline{Y}, \overline{M}, \overline{V}; where the vehicle-treated condition is V, single compounds are X or Y and the multiplexed combination is M. The synergetic factor (SP) is considered as the ratio between the vehicle subtracted multiplexed gene expression and t¹ vehicle subtracted additive gene expression, hence

$$SF = \frac{\overline{M} - \overline{V}}{( \overline{X} - \overline{V} ) + ( \overline{Y} - \overline{V} )} .$$

Results

H-4-II-E rat liver cells were stimulated with either vehicle (0.1% DMSO), 50 μM EGCG, 1 μM biotin, 50 μM cysteamin, applied as single compounds, as well as the combinations of EGCG/biotin, and cysteamin/biotin for 24 h. Total RNA samples were processed to cRNA probes and hybridized to Affymetrix rat 230 (SubA) arrays. Calculations of the raw fluorescence data was carried out in the Affymetrix MAS 4.0 program. The MeanAvgDiff expression levels, standard deviations (SD) and the values of the SF for the glucose-6-phosphatase gene expression were picked from a filtered list of genes generated in the RACE-A program.

TABLE 2

| | MeanAvgDiff | | |
|---|---|---|---|
| Compound(s) | (Mean Relative Expression) | SD | SF |
| V (Vehicle) | 535.01 | 96.24 | — |
| A (EGCG) | 484.8 | 34.36 | — |
| B (Biotin) | 516.66 | 60.82 | — |
| C (Cysteamin) | 447.42 | 18.69 | — |
| D (EGCG/Biotin) | 332.46 | 87.21 | 2.95* |
| E (Cysteamin/Biotin) | 343.91 | 59.85 | 1.80* |

*p < 0.05 (unpaired student's t-test)

Gene expression levels of the catalytic subunit of glucose-6-phosphatase (Affymetrix Id: 1370725_a_at) were determined using the Affymetrix GeneChip method. Mean average differences (MeanAvgDiff) were calculated by pair-wise comparisons of replicate samples.

The activity of Glc6Pase, which enables the liver to produce glucose, is increased during short-term fasting, in agreement with the enhancement of liver gluconeogenesis. On the other hand, GK activity, which allows the liver to utilize glucose, is decreased during fasting. In the fed state, the mechanisms of short-term regulation of the activity of both enzymes takes place during the postprandial period. Overproduction of glucose by the liver is the major cause of fasting hyperglycemia in both insulin-dependent and non-insulin-dependent diabetes mellitus. The distal enzymatic step in the process of glucose output is catalyzed by the glucose-6-phosphatase complex. As can be seen from Table 2, H-4-II-E cells treated with either EGCG/biotin or cysteamin/biotin show a unexpected synergistic decrease in the messenger RNA of the catalytic subunit of glucose-6-phosphatase. Accordingly, the data of Table 2 show that biotin surprisingly acts in a synergistic manner with EGCG or the pantethine metabolite cysteamin in down-regulating the expression of one of the major glucose-metabolism rate-limiting enzymes in rat liver cells. Consequently, such a combination treatment will reduce the glucose output and will therefore reduce hyperglycemia and prevent diabetes.

1. A composition comprising biotin in an amount sufficient to administer to a subject a daily dosage of 0.01 mg per kg body weight to about 3 mg per kg body weight and at least one additional component selected from the group consisting of pantethine or a metabolite thereof, EGCG, phytanic acid, lipoic acid and policosanol.

2. A composition according to claim 1 comprising biotin and at least one additional component selected from the group consisting of pantethine or a metabolite thereof, EGCG, phytanic acid and lipoic acid.

3. A composition according to claim 1 wherein pantethine is present.

4. A composition as in claim 3 containing pantethine in an amount sufficient to administer to a subject a daily dosage of 1 mg per kg body weight to about 50 mg per kg body weight.

5. A composition according to claim 1 wherein EGCG is present.

6. A composition as in claim 5 containing EGCG in an amount sufficient to administer to a subject a daily dosage of 0.3 mg per kg body weight to about 30 mg per kg body weight.

7. A composition according to claim 1 wherein phytanic acid is present.

8. A composition as in claim 7 containing phytanic acid in an amount sufficient to administer to a subject a daily dosage of 1 mg per kg body weight to about 100 mg per kg body weight.

9. A composition according to claim 1 wherein lipoic acid is present.

10. A composition as in claim 9 wherein lipoic acid is present in an amount sufficient to administer to a subject a daily dosage of 0.3 mg per kg body weight to about 30 mg per kg body weight.

11. A composition according to claim 1 wherein policosanol is present.

12. A composition as in claim 11 wherein policosanol is present in an amount sufficient to administer to a subject a daily dosage of 0.002 mg per kg body weight to about 1.5 mg per kg body weight.

13. A composition according to claim 1 which is in dosage unit form.

14. A composition as in claim 13 wherein the dosage unit form is a solid dosage unit form.

15. A composition as in claim 14 wherein the dosage unit form contains about 0.35 mg to about 200 mg of biotin.

16. A composition as in claim 13 wherein the dosage unit form is a liquid dosage unit form.

17. A composition as in claim 16 wherein the dosage unit form contains about 0.35 mg to about 200 mg of biotin per ml.

18. A composition according to claim 1 which is a food or beverage or a supplement composition for a food or beverage.

19. A food or beverage comprising about 0.03 mg to about 50 mg of biotin per serving and at least one additional component selected from the group consisting of pantethine or a metabolite thereof, EGCG, phytanic acid, lipoic acid and policosanol.

20. A food or beverage comprising about 0.03 mg to about 50 mg of biotin per serving and at least one additional component selected from the group consisting of pantethine or a metabolite thereof, EGCG, phytanic acid and lipoic acid.

21-25. (canceled)

26. A method for the treatment of both type 1 and 2 diabetes, and for the prevention of type 2 diabetes in those individuals with pre-diabetes, or impaired glucose tolerance (IGT) or obesity which comprises administering to a subject in need of such treatment biotin in a daily dosage of 0.01 mg per kg body weight to about 3 mg per kg body weight together with at least one additional component selected from the group consisting of pantethine or a metabolite thereof, EGCG, phytanic acid, lipoic acid and policosanol.

27. A method for the treatment of both type 1 and 2 diabetes, and for the prevention of type 2 diabetes in those individuals with pre-diabetes, or impaired glucose tolerance (IGT) or obesity which comprises administering to a subject in need of such treatment biotin in a daily dosage 0.01 mg per kg body weight to about 3 mg per kg body weight together with at least one additional component selected from the group consisting of pantethine or a metabolite thereof, EGCG, phytanic acid and lipoic acid.
